(19) United States
(12) Patent Application Publication
(10) Pub. No.: US 2009/0071230 A1
(43) Pub. Date: Mar. 19, 2009

(54) Method for reliable, individualized measurement and warning of air pollution, and associated device

(76) Inventor: Bruno Aubert, Connaux (FR)

Correspondence Address: Bruno Aubert, 250 route de Cavillargues, CONNAUX, 30330, omitted

(21) Appl. No.: 12/159,089

(22) PCT Filed: Dec. 22, 2006
(86) PCT No.: PCT/FR2006/002848

§ 371 Date: Sep. 12, 2008

(30) Foreign Application Priority Data
Dec. 26, 2005 (FR) 0513398

(51) Int. Cl. G01N 33/00; G08B 21/14

(52) U.S. Cl. 73/31.02; 340/632

(57) ABSTRACT

The method consists in, first, circulating the air in which the content of a specific pollutant is to be measured through or over materials that reversibly absorb compounds in the air that interfere with the measurement of pollution. These absorbent materials are employed in the form of self-fixing patches or membranes that can be disposed of after use and are placed between the air to be measured and the measurement means. These patches or membranes can be used for all types of measurement means, such as electronic sensors or calorimetric indicators (in which case the membrane will advantageously be transparent). This actual pre-treatment of the air will thus allow reliable, reproducible measurement irrespective of the environmental conditions (humidity, presence of other pollutants, etc.). For humidity, for example, the membrane will consist of silica gel or cotton wool. A first application is an individualized warning device that can be carried on the reverse of a jacket or on the wrist, for alerting asthmatics to the presence of a peak in ozone pollution or sulphur and nitrogen oxide pollution.

### FIELD OF THE INVENTION

The present invention relates to a method for measuring and alerting of air pollution peaks to warn a population, in particular the asthmatics, of the potential risks of being exposed to polluted air, and its associated device.

Said method is fit, as its description will show, to detect in its immediate surroundings, the pollutants present in the air that we breathe whose concentration would exceed the levels characterized as dangerous by health services, and to provide individualized alerts.

It is well known that persons suffering from respiratory ailments, as in the case of the asthmatics, must, in presence of air pollutants, limit their outside activities when certain levels of air pollution have been surpassed and in many cases refrain to engage in more demanding physical activities.

Until now, when an asthmatic wanted to know of the risks of going outside in regard to levels of air pollutants such as ozone, nitrogen or sulfur oxides, she/he had to rely on different medias (television, radio . . . ) without being sure that a pollution peak would not sporadically occur later in the day.

In addition, when the person is isolated from the sources of information, no other prevention solution is available.

Apparatus in the form of ozone detectors and monitors, sulfur and nitrogen detectors and monitors exists on the market, notably based on spectrometers, but their large size limit their usage and they are definitely not wearable. Furthermore, their complexity makes it impossible to be operated by children.

Small sampling devices can also be found but they require further analysis of the samples, usually performed at a later date at a specialized laboratory.

Finally, small pollution detectors and monitors based on electronic sensors and colorimetric indicators that change color when a specific pollutant is present are available, but numerous parameters such as humidity (an ozone absorbent) can disrupt the measurement in a significant way (a 1 to 10 factor) and make it impossible to effectively and reliably warn sensitive persons.

The present invention aims to eliminate the principal problem of persons and notably asthmatics not being aware of the presence a potential risk with a method and a miniature device, reliable and easy to operate that will undoubtedly provide an alert when unhealthy pollutants levels have been reached.

The method of the present invention consists in a process where the air targeted to have its concentration in a predetermined pollutant measured, is beforehand circulated through or over materials capable of absorbing in a reversible way the air compounds that interfere in measuring pollution.

These absorbent materials are implemented in the forms of patches or porous membranes that are self-attaching by magnetic, mechanical, reversible glue, or self-grabbing fabric and are disposable after usage (in a preventive way such as daily, weekly or monthly depending on the quantity of absorbent used or as soon as the porosity has been compromised by pollutants and dust, detectable by a loss of discharge pressure at the device air pump level) In the case where air is circulated through the patch or membrane, the various compounds absorbing materials can be arranged in a multi layered form. In the case where air is circulated over the patch or membrane, the same absorbent materials will be arranged in a single layer.

For example, one of the materials used in the patch or the porous membrane could be silica gel that absorbs humidity or hydrophilic cotton. The measure of ozone concentration for example will be accurate in dry conditions as well as under rainy conditions.

To absorb nitrogen or sulfur oxides, specifically activated carbons, targeting these molecules will be used.

In addition, these absorbing materials must be reversible to permit the liberation of all or a fraction of the gases that they have absorbed as they move in an environment with a smaller concentration of the absorbed gas to help the regeneration of the absorbing material and also to smooth the variations in interfering gas concentrations.

In the case of humidity, a hydrophilic material such as cotton will absorb the excess of humidity as one moves from an outside location (where a humidity level of 10% is not uncommon in winter) to an inside location (where humidity level can reach 90%).

As soon as the person moves back outside, cotton will free its humidity to make the change in air humidity on the sensor more progressive.

Just like that, this genuine air pretreatment ensures the same optimal measuring conditions for the electronic sensor or the calorimetric indicator regardless of atmospheric conditions.

A visual alarm, vibrating or ringing, reliable will be provided by an electronic board also responsible for managing the different alert levels. Advantageously, a hydrophobic lining will be positioned between the air to be analyzed and the patch or membrane (on the most exterior layer) to permit operation in the rain or when hands are being washed.

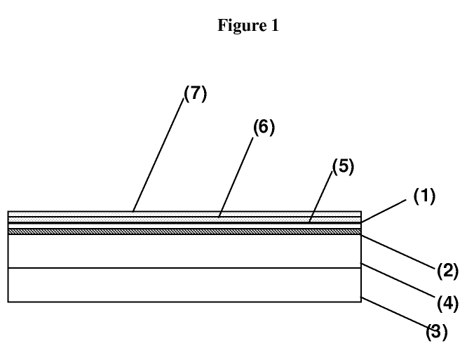

FIG. 1 shows a schematic cross-sectional view of an example of the associated device of the invention with a colorimetric indicator and a micro porous membrane that permit air circulation through it.

The micro porous transparent membrane (1) is made of various layers, also micro porous and transparent, and of materials that absorb air compounds that interfere with the measure of air pollution. The said membrane is directly set on a colorimetric indicator, itself micro porous (2).

The first layer (7) is a hydrophobic material to permit operation under rainy conditions.

The second layer (6) is an NO2 and SO2 absorbent primarily made of activated carbon.

The third layer (5) consists of a humidity absorbent made of silica gel.

A piezoelectric micro-pump (4) connected to a battery (3) ensures a continuous and steady flow through the membrane and the micro porous colorimetric indicator.

Monitoring the intensity supplying the pump can reveal if the patch or the membrane are obstructed.

In the case where an electronic sensor is used, the patch or the micro porous membrane can be placed directly on the pump that circulates the processed air toward the sensor.

The patch or the membrane can also be non porous. In that case a small space is fitted between the patch and the pump to permit that the air directed toward the sensor circulates over the whole patch or membrane surface to rid it of its interferents.

The sensor is itself also connected to an electronic circuit that will set off a LED, a LCD, a vibrating device or a buzzer when specifics levels have been reached.

Components miniaturization such as the piezoelectric pumps made possible to design a device no larger than a one euro coin.

A first application is a personal alert device, wearable on the lapel or at the wrist, which warns asthmatics of the presence of ozone or nitrogen and sulfur oxides pollution peaks.

Periodically, the end user replaces the absorbing patch or membrane on its measuring device to make sure that he/she will be warned in the case of an eventual pollution.

1. A method for measuring air pollution and providing reliable and individualized alerts, characterized in that before the measure permitting said alerts is taken, air compounds interfering with said measure are removed by specifically selected absorbent materials in the forms of self-attaching patches or membranes, disposable after usage and set between air to be measured and the measuring means.

2. A method for measuring air pollution and providing reliable and individualized alerts according to claim 1 characterized in that said specifically selected absorbent materials are reversible.

3. A device for carrying out the process according to claim 1 characterized in that one of the specific materials absorbing said air compounds interfering with the measure of air pollution is a hydrophilic material such as silica gel or cotton.

4. A device for carrying out the process according to claim 1 characterized in that one of the specific materials absorbing said air compounds interfering with the measure of air pollution is a material that is capable of absorbing nitrogen oxides.

5. A device for carrying out the process according to claim 1 characterized in that one of the specific materials absorbing said air compounds interfering with the measure of air pollution is a material that is capable of absorbing sulfur oxides.

6. A device for carrying out the process according to any one of claims 2-4 characterized in that a hydrophobic material is set between the air to be measured and the patches or membranes.

7. A device for carrying out the process according to any one of claims 2-5 characterized in that an air pump ensures a steady flow of air to be measured toward the patches and the measuring means.

8. A device for carrying out the process according to any one of claims 2-6 characterized in that said measuring means are connected to an electronic circuit that manages the various alerts in accordance with pollutants level regulations.

9. A device for carrying out the process according to any one of claims 2-7 characterized in that said electronic circuit permits the detection of an increasing drop of pressure at the patch or membrane level and alerts the user to replace said patch or membrane.

10. A device according to any one of the preceding claims characterized in that it comprises a micro pump, a pollution sensor, an electronic circuit that manages said alerts and a patch covered with materials capable of absorbing air pollution measurement interferents and implemented to alert individuals that unhealthy pollution levels have been reached.
